(19) United States
(12) Patent Application Publication
(10) Pub. No.: US 2014/0248221 A1
(43) Pub. Date: Sep. 4, 2014

(54) COMPOSITIONS FOR ORAL USE AND METHODS FOR TREATING THE ORAL MUCOSA, LIPS, AND PERIORAL REGIONS

(71) Applicant: JR CHEM, LLC, Key West, FL (US)

(72) Inventor: Jose E. Ramirez, Key West, FL (US)

(73) Assignee: JR CHEM, LLC, Key West, FL (US)

(21) Appl. No.: 14/276,323

(22) Filed: May 13, 2014

Related U.S. Application Data
(63) Continuation of application No. 13/158,820, filed on Jun. 13, 2011.

(51) Int. Cl. A61K 8/73; A61K 8/58; A61Q 19/08; A61Q 11/00

(52) U.S. Cl. CPC A61K 8/73; A61Q 11/00; A61K 8/58; A61Q 19/08
USPC 424/49; 514/494; 514/54

(57) ABSTRACT

Compositions containing polymetal complexes useful in treating disorders of the oral mucosa, lips, and perioral regions are described.

### BACKGROUND

1. Technical Field

The present disclosure relates to compositions for oral use and methods for treating the oral mucosa, lips, and perioral regions. More particularly, the compositions for oral use include organic compounds containing at least two elements selected from calcium, copper, magnesium, silver, gold, aluminum, scandium, titanium, vanadium, chromium, manganese, iron, cobalt, nickel, zinc, gallium, germanium, yttrium, zirconium, niobium, molybdenum, technetium, ruthenium, rhodium, palladium, cadmium, indium, selenium, and tin. The organic compounds can be prepared by reacting a polyfunctional compound with two or more of the listed elements.

2. Background

A variety of disorders affect the oral mucosa, lips, and perioral region of the face. The oral mucosa is the mucous membrane epithelium of the mouth. It can be divided into three categories—the masticatory mucosa, the lining mucosa, and the specialized mucosa. The lips surround the mouth and the entrance of the oral cavity, and the perioral region includes the skin surrounding the lips and mouth.

Medical conditions in this area can be problematical, inconvenient to treat, and painful to endure. The most common medical condition is cheilitis which involves the inflammation of the lip. The types of cheilitis are exfoliative, allergic, actinic, glandular, bacterial, and others. Other common superficial lesions of the oral mucosa, lips, and perioral region include candidiasis, recurrent herpes labialis, recurrent aphthous stomatitis, erythema migrans, hairy tongue, and lichen planus. Recognition and diagnosis require taking a thorough history and performing a complete oral examination. Knowledge of clinical characteristics such as size, location, surface morphology, color, pain, and duration is helpful in establishing a diagnosis.

Oral candidiasis may present as pseudomembranous candidiasis, glossitis, or perlèche (angular cheilitis). Oral candidiasis is common in infants, but in adults it may signify immune deficiency or other illness. Herpes labialis typically is a mild, self-limited condition. Recurrent aphthous stomatitis most often is a mild condition; however, severe cases may be caused by nutritional deficiencies, autoimmune disorders, or human immunodeficiency virus infection. Erythema migrans is a waxing and waning disorder of unknown etiology. Hairy tongue represents elongation and hypertrophy of the filiform papillae and most often occurs in persons who smoke heavily. Oral lichen planus is a chronic inflammatory condition that may be reticular or erosive. Certain risk factors have been associated with each of these lesions, such as poor oral hygiene, age, tobacco use, and alcohol consumption, and some systemic conditions may have oral manifestations. Many recommended therapies for oral lesions are unsupported by randomized controlled trials.

The treatments recommended for common Superficial Oral Lesions include: ointments for cheilitis exfoliative; sunblocks and sunscreens for actinic cheilitis; corticosteroids for allergic cheilitis; ointments and moisturizers for cheilitis glandularis; topical antifungals (e.g., nystatin [Mycostatin] suspension or troches, fluconazole [Diflucan], or systemic antifungals) for candidiasis-cheilatis angularis or perleche; topical agents that include 1% penciclovir cream and systemic agents (e.g., acyclovir, famciclovir) for recurrent herpes labialis; fluocinonide gel (Lidex) or triamcinolone acetonide (Kenalog in Orabase), amlexanox paste (Aphthasol), and/or chlorhexidine gluconate (Peridex) mouthwash for recurrent aphthous stomatitis; topical corticosteroids, zinc supplements, or topical antimicrobial rinses for erythema migrans (central erythemal surrounded by white to yellow borders on tongue or lips); and antimicrobials like 10% carbamide peroxide for hair tongue.

Other medical conditions, such as chronological aging and/or environmental damage to the oral mucosa, lips, and perioral region can only be treated with rejuvenation procedures which can be both costly and painful, requiring multiple visits to a dermatologist along with the use of anesthesia or other medications. The two most common procedures are neuromuscular toxins and soft tissue fillers.

Neuromuscular toxins, such as Botulinum Toxin type A, block nerve signals that cause muscles to contract. The toxin works directly where it is placed, and thus can be artistically used to alter facial expressions. Botox Cosmetic® is widely recognized, and was the first neurotoxin to be approved for cosmetic use in the United States. Other manufacturers are producing variant toxins that will likely be approved for use in the near future, including Reloxin and PurTox. These toxins will be differentiated by their time to onset, duration of effect (the clinical effects of Botox Cosmetic® are typically 3 to 4 months), and the distance of effect from the injection site.

Soft tissue fillers restore volume to the face and can add structure as well. Depending on the type of filler and the depth at which it is injected, fine lines on the surface of the skin can be smoothed out, deep lines (e.g., nasolabial folds) may be filled out, and soft tissue (e.g., lips) and even facial bone structure can be augmented.

Complications may arise when undergoing surgical rejuvenation. Common reactions can range from redness, swelling or itching at the injection site(s), bleeding, uneven lips, movement of the implanted filler, or extrusion if the implant breaks through the outermost surface of the skin. The usual expected reactions include swelling and bruising that can last from several days to a week. In addition, many injectables have a short-lived effect (from 9-12 weeks). Fat injections provide longer-lasting results, but are sometimes unpredictable as to the degree of improvement, may have lumping or scarring effect, and must be harvested from another part of the body.

It would be highly desirable to develop new methods and compositions that can solve the need for painful and costly surgery.

None of the approaches to medical conditions of the oral region include the oral use of a composition which contains at least one polymetal complex as described herein.

### SUMMARY

Accordingly, the compositions for oral use which contain at least one polymetal complex are described in the present disclosure. The present disclosure relates to compositions for oral use and methods for treating oral disorders. More particularly, the compositions for oral use include organic compounds containing at least two elements selected from calcium, magnesium, copper, silver, gold, aluminum, scandium, titanium, vanadium, chromium, manganese, iron, cobalt, nickel, zinc, gallium, germanium, yttrium, zirconium, niobium, molybdenum, technetium, ruthenium, rhodium, palladium, cadmium, indium, selenium, and tin. The organic compounds can be prepared by reacting a polyfunctional compound with two or more listed elements. Methods of making such reaction products are also described.

The synthetic compositions are prepared for oral administration and may take the form of any gas, liquid, solid, or combination thereof which is capable of being administered to the oral region of a subject. In embodiments, the compositions may also include suitable materials which allow the compositions to take the form of useful oral delivery devices such as sprays, gels, creams, ointments, foams, aerosols, lipsticks, and the like.

In addition, the present disclosure describes methods for treating oral disorders which includes administering to the oral region of a subject in need of such treatment an effective amount of the compositions described herein.

In embodiments, compositions of the present disclosure allow for the treatment of the perioral region without needles or scalpels. In some treatment applications, oral use of the compositions of the present disclosure improves appearance from the inside out, resulting, for example, in a substantial reduction of wrinkles around the mouth. The inside of the lip is made up of oral mucosa that is a network of empty tubes and chambers. As aging occurs the salivary glands clog and shrink. While not wishing to be bound by any theory, it is believed that when a composition of the present disclosure is applied inside the oral cavity, such as the inner lips, the sweat glands are induced to secrete more moisture and help clear the salivary ducts. The moisture will fill the tubular glands that will empty on the surface of the lips and create fuller, smoother looking lips.

### DETAILED DESCRIPTION

The present disclosure describes compositions for oral use which include at least one polymetal complex. The synthetic compositions are prepared for administration to the oral cavity and may be used in methods for treating disorders of the oral region.

As defined herein, the term “oral region” is meant to include the masticatory mucosa, lining mucosa, and specialized mucosa of the oral mucosa, lips, and perioral region of a subject, including the lower third of the face.

The term “disorder” or “oral disorder” is meant to include any condition that causes an irregularity or detectable change in the oral region due to a number of factors, such as a diseased or dysfunctional state, chronological aging, and/or environmental damage. Non-limiting examples of such disorders include those conditions described above, as well as dryness, itching, thinning, thickening, wrinkling, including both fine superficial wrinkles and coarse deep wrinkles, skin lines, crevices, bumps, large pores, scaliness, flakiness, and/or other forms of skin unevenness or roughness. Such disorders further include undesirable tactile conditions such as loss of skin elasticity, sagging, loss of skin firmness, loss of skin tightness, and/or loss of skin recoil from deformation. It is understood, that the listed disorders are non-limiting and that only a portion of the conditions suitable for treatment in accordance with the present disclosure are listed herein.

The terms “treatment” and “treating” are meant to include, but not be limited to, changes in the subject's status. The changes may be either subjective or objective and may relate to features such as symptoms or signs of the disease or disorder receiving therapy. For example, if the patient notes reduced discomfort or decreased pain, then successful treatment has occurred. Similarly, if the clinician notes objective changes, such as by histological analysis of a biopsy sample, then treatment has also been successful. Alternatively, the clinician may note a decrease in the size of lesions or other abnormalities upon examination of the patient. This would also represent an improvement or a successful treatment. Prevention of deterioration of the subject's status is also included by the term.

The term “subject” as used herein includes animals, such as a mammal, including a human.

The term “effective amount” means a dosage sufficient to produce a desired result. The desired result may comprise a subjective or objective improvement in the recipient of the dosage.

As described herein, a new approach for treating oral disorders includes administering to a subject in need of such treatment an effective amount of a composition which includes at least one polymetal complex. The polymetal complex can be the reaction product of a polyfunctional compound with two or more elements. The preparation of reaction products of polyfunctional compounds with two or more elements, and compositions containing such reaction products, are described.

The polyfunctional compound can be any compound that contains at least two functional groups that may complex with metal cations in solution. Among the functional groups that may be present include carboxylic acid groups and amino groups. Suitable polyfunctional compounds include, but are not limited to polyfunctional acids, polyfunctional amines, and amino acids. Other suitable polyfunctional compounds will be readily envisioned by those skilled in the art reading the present disclosure. It should of course be understood that mixtures of polyfunctional compounds may be used.

Polyfunctional acids are primarily compounds having two or more carboxylic acid groups. Non-limiting examples of polyfunctional acids include maleic acid, fumaric acid, citraconic acid, itaconic acid, glutaconic acid, phthalic acid, isophthalic acid, terephthalic acid, cyclohexane dicarboxylic acid, citric acid, succinic acid, adipic acid, sebacic acid, azealic acid, malonic acid, dodecanedioic acid, 1,18-octadecanedioic acid, dimer acids (prepared from a mono-, di- or triunsaturated fatty acid, acid wax, acid anhydride grafted wax, or other suitable polycarboxylic acid reacting compound), and alkenyl succinic acids (such as n-dodecenylsuccinic acid, docecylcucinic acid and octadecenylsuccinic acid). The polyfunctional acid can be present in acidic form, anhydride form, ionic form, salt form, or mixtures thereof.

It is also contemplated that the polyfunctional acid can be a naturally occurring or synthetic polymer that includes two or more functional groups per polymer molecule, such as, for example, two or more carboxylic acid groups. One such polymeric polyfunctional acid is hyaluronic acid, a polymer of disaccharides, themselves composed of D-glucuronic acid and D-N-acetylglucosamine, linked via alternating β-1,4 and β-1,3 glycosidic bonds. Hyaluronic acid has a large number of carboxylic acid groups available which can readily interact with a plurality of different elements. Another naturally occurring polycarboxilic acid is bacitracin. Bacitracin is an antibiotic that will react with zinc and copper ions to form a cu-zn bacitrate bimetallic complex. Cu-zn bacitrate provides a greater zone of inhibition activity than zn bacitracin.

Amino acids may also be used as the polyfunctional compound. Amino acids are known to those skilled in the art and include at least a carboxylic acid functionality and an amino functionality. Suitable amino acids include naturally occurring amino acids and synthetic amino acids. Non-limiting examples of amino acids include, but are not limited to: aminopolycarboxylic acids (e.g., aspartic acid, β-hydroxyaspartic acid, glutamic acid, β-hydroxyglutamic acid, β-methylaspartic acid, β-methylglutamic acid, β,β-dimethylaspartic acid, γ-hydroxyglutamic acid, β,β-dihydroxyglutamic acid, β-phenylglutamic acid, γ-methyleneglutamic acid, 3-aminoadipic acid, 2-aminopimelic acid, 2-aminosuberic acid, and 2-aminosebacic acid); amino acid amides such as glutamine and asparagine; polyamino- or polybasic-monocarboxylic acids such as arginine, lysine, β-aminoalanine, γ-aminobutyrine, ornithine, citruline, homoarginine, homocitrulline, hydroxylysine, allohydroxylsine, and diaminobutyric acid; other basic amino acid residues such as histidine; diaminodicarboxylic acids such as α,α′-diaminosuccinic acid, α,α′-diaminoglutaric acid, α,α′-diaminoadipic acid, α,α′-diaminopimelic acid, α,α′-diamino-β-hydroxypimelic acid, α,α′-diaminosuberic acid, α,α′-diaminoazelaic acid, and α,α′-diaminosebacic acid; imino acids such as proline, hydroxyproline, allohydroxyproline, γ-methylproline, pipecolic acid, 5-hydroxypipecolic acid, and azetidine-2-carboxylic acid; mono- or di-alkyl (typically C₁-C₈ branched or normal) amino acids such as alanine, valine, leucine, allylglycine, butyrine, norvaline, norleucine, heptyline, α-methylserine, α-amino-α-methyl-γ-hydroxyvaleric acid, α-amino-α-methyl-δ-hydroxyvaleric acid, α-amino-α-methyl-ε-hydroxycaproic acid, isovaline, α-methylglutamic acid, α-aminoisobutyric acid, α-aminodiethylacetic acid, α-aminodiisopropylacetic acid, α-aminodi-n-propylacetic acid, α-aminodiisobutylacetic acid, α-aminodi-n-butylacetic acid, α-aminoethylisopropylacetic acid, α-amino-n-propylacetic acid, aaminodiisoamyacetic acid, α-methylaspartic acid, α-methylglutamic acid, 1-aminocyclopropane-1-carboxylic acid, isoleucine, alloisoleucine, tert-leucine, β-methyltryptophan and α-amino-β-ethyl-β-phenylpropionic acid; β-phenylserinyl; aliphatic α-amino-β-hydroxy acids such as serine, β-hydroxyleucine, β-hydroxynorleucine, β-hydroxynorvaline, and α-amino-β-hydroxystearic acid; α-Amino, α-, γ-, δ- or ε-hydroxy acids such as homoserine, γ-hydroxynorvaline, δ-hydroxynorvaline, and epsilon-hydroxynorleucine residues; canavine and canaline; γ- hydroxyornithine; 2-hexosaminic acids such as D-glucosaminic acid or D-galactosaminic acid; α-Amino-β-thiols such as penicillamine, β-thiolnorvaline, or β-thiolbutyrine; other sulfur containing amino acid residues including cysteine, homocystine, β-phenylmethionine, methionine, S-allyl-L-cysteine sulfoxide, 2-thiolhistidine, cystathionine, and thiol ethers of cysteine or homocysteine; phenylalanine, tryptophan and ring-substituted α amino acids such as the phenyl- or cyclohexylamino acids α-aminophenylacetic acid, aaminocyclohexylacetic acid, and α-amino-β-cyclohexylpropionic acid; phenylalanine analogues and derivatives comprising aryl, lower alkyl, hydroxy, guanidino, oxyalkylether, nitro, sulfur or halo-substituted phenyl (e.g., tyrosine, methyltyrosine and o-chloro-, p-chloro-, 3,4-dicloro, o-, m- or p-methyl-, 2,4,6-trimethyl-, 2-ethoxy-5-nitro-, 2-hydroxy-5-nitro-, and p-nitrophenylalanine); furyl-, thienyl-, pyridyl-, pyrimidinyl-, purinyl- or naphthylalanines; tryptophan analogues and derivatives including kynurenine, 3-hydroxykynurenine, 2-hydroxytryptophan, and 4-carboxytryptophan; α-Amino substituted amino acids including sarcosine (N-methylglycine), N-benzylglycine, N-methylalanine, N-benzylalanine, N-methylphenylalanine, N-benzylphenylalanine, N-methylvaline, and N-benzylvaline; and α-Hydroxy and substituted α-hydroxy amino acids including serine, threonine, allothreonine, phosphoserine and phosphothreonine, glycine, alanine, valine, leucine, isoleucine, serine, threonine, cysteine, methionine, glutamic acid, aspartic acid, lysine, hydroxylysine, arginine, histidine, phenylalanine, tyrosine, tryptophan, proline, asparagine, glutamine, and hydroxyproline. Polyaminoacids may also be used provided they form complexes with the elements employed.

The organic compounds can be prepared by reacting a polyfunctional compound with two or more elements. The elements can be chosen from calcium, magnesium, copper, silver, gold, aluminum, scandium, titanium, vanadium, chromium, manganese, iron, cobalt, nickel, zinc, gallium, germanium, yttrium, zirconium, niobium, molybdenum, technetium, ruthenium, rhodium, palladium, cadmium, indium, selenium, and tin. Those skilled in the area will readily envision suitable compounds for providing the elements in solution.

In embodiments, a bimetal complex is formed by an aqueous solution containing: a) one or more polycarboxylic acids, b) one or more polyamines, and/or c) one or more amino acids having at least two carboxylic acid groups with two or more elements selected from calcium, magnesium, copper, silver, gold, aluminum, scandium, titanium, vanadium, chromium, manganese, iron, cobalt, nickel, zinc, gallium, germanium, yttrium, zirconium, niobium, molybdenum, technetium, ruthenium, rhodium, palladium, silver, cadmium, indium, and tin.

For example, water soluble salts containing the element may be used. The salts may be organic or inorganic. Suitable water-soluble silver salts include silver nitrate, silver acetate, silver propionate, silver sulfate, silver butyrate, silver isobutyrate, silver benzoate, silver tartrate, silver salicylate, silver malonate, silver succinate, and silver lactate. Suitable water-soluble aluminum salts include aluminum potassium sulfate, aluminum chloride, aluminum sodium sulfate, aluminum sodium phosphate, aluminum sulfate, aluminum nitrate, and sodium aluminate. Suitable water-soluble copper salts include copper sulfate, fluoroborate, hydroxide, borate, fluoride, carbonate, oxychloride, formate or acetate. Suitable water-soluble zinc salts include zinc chloride, zinc bromide, zinc iodide, zinc chlorate, zinc bromate, zinc chlorite, zinc perchlorate, zinc sulfate, zinc nitrate, zinc nitrite, zinc borate, zinc metaborate, basic zinc borate, zinc hexafluorosilicate, zinc hypophosphite, zinc glycerophosphate, zinc bichromate, zinc citrate, zinc thionate, zinc dithionate, zinc tetrathionate, zinc pentathionate, zinc thiocyanate, zinc benzoate, zinc acetate, zinc salicylate, zinc picrate, zinc permanganate, zinc hydrogen phosphate, zinc formate, zinc ethylsulfate, and zinc phenolsulfonate. Examples of suitable water soluble nickel salts that may be used include nickel sulfate hexahydrate and nickel chloride hexahydrate. It should be understood that the listed salts are only a small portion of the salts suitable for use in accordance with the present disclosure. For example, inorganic salts are suitable provided that they provide coordination element cations when placed in an aqueous solution. Thus, the foregoing list of salts should be considered a non-limiting, illustrative list.

For carrying out the process, a reaction solution can be prepared by mixing the various ingredients in water. Water in the mixture may advantageously be added in limited amounts sufficient to allow the reaction product to precipitate from solution upon formation. Accordingly, the reaction mixture is not so dilute as to prevent product precipitate formation. Where necessary, mixing and heating can be used to bring the reactants to about 40° C.-100° C. in order to force the reaction. As a result, reactant solubility may be enhanced through energy input such as microwave heating or addition of boiling water. The input of the energy may take place through any instrument capable of heating the aqueous reaction mixture. The reaction products formed in solution may be immediately separated so that their production can take place in a continuous process. Where a short reaction time and rapid crystallization of the reaction product occur, the conversion may be carried out continuously, and the recovery of the resultant solid product may take place by any conventional manner such as filtering, centrifugation, or sedimentation.

In embodiments, the method of forming a polymetal complex includes forming a solution by adding to a solvent (i) at least one polyfunctional compound selected from polycarboxylic acids, polyamines, and amino acids having at least two carboxylic acid groups, and (ii) basic salts of two or more elements selected from one or more of calcium, magnesium, copper, silver, gold, aluminum, scandium, titanium, vanadium, chromium, manganese, iron, cobalt, nickel, zinc, gallium, germanium, tin, yttrium, zirconium, niobium, molybdenum, technetium, ruthenium, rhodium, palladium, silver, cadmium, and indium; and recovering a polymetal complex that includes the two or more elements joined to a central unit derived from the polyfunctional compound.

The polyfunctional compound is present in the reaction mixture in amounts that will contact metal cations in an aqueous solution. Suitable amounts of polyfunctional compound also include excess amounts in relation to the amount of metal cations. In embodiments, polyfunctional compound is present in a 3:1:1 molar ratio in relation to the metal constituents. In embodiments, the polyfunctional compound is malonic acid which can be present in acidic form, salt form, or mixtures thereof. In embodiments, the process parameters are especially advantageous if the polyfunctional compound is added to excess in comparison to the metal counter cation constituents. Depending on the desired complex, the latter are added so that the molar ratio of polyfunctional compound to metal ions is approximately 3:2.

For metallic cations that are in monometallic substitution reactions with polyfunctional salts, the ratios could be altered to increase the water solubility of the bimetallic salts. For example, if the bimetallic complex includes magnesium and calcium, the molar ratio of the polyfunctional compound to metal ions may still be approximately 3:2, however, to increase the water solubility of the complex the ratio of the presence of polyfunctional compound to the metal constituents may change to a 3:1.9:0.1 molar ratio, for example, the 1.9 being the most soluble metallic cation, i.e., magnesium, and 0.1 being the highly insoluble component, i.e., calcium.

In embodiments, the elements may be present as one or more ionic compounds formed by joining one or more independent element molecules or ions of a first type and one or more element molecules or ions of a second type to a central unit by ionic bonds. For example, the reaction product may be in the form of a trinuclear cation, where structurally independent cation metal hydrates are bridged by a central unit. However, various valence modes are possible depending on the source of the elements and synthesis conditions. In embodiments, the central unit may be a multi-membered ring such as eight-membered ring, six-membered ring, and four-membered metalocycle for bridging or bonding functions between the constituent elements. Accordingly, the crystal structures of the reaction products can be very diverse, from ionic to three-dimensional polymers. In embodiments, the reaction products are present in several hydrate, and polymorphic forms.

In embodiments, the polymetal complex includes one or more molecules of a first element, one or more molecules of a second element different from the first element, and a central unit, wherein the central unit includes at least one compound selected from polycarboxylic acids, polyamines, and amino acids having at least two carboxylic acid groups and the center unit bridges the one or more molecules of a first element and one or more molecules of a second element by chemical bonding.

In embodiments, the polymetal complex is formed by bonding a) at least one polyfunctional compound selected from polycarboxylic acids, polyamines, and amino acids having at least two carboxylic acid groups with b) basic salts of two or more elements selected from one or more of calcium, magnesium, copper, silver, gold, aluminum, scandium, titanium, vanadium, chromium, manganese, iron, cobalt, nickel, germanium, zinc, gallium, yttrium, zirconium, niobium, molybdenum, technetium, ruthenium, rhodium, palladium, cadmium, selenium, tin, and indium, wherein the bonding includes two or more elements joined to a central unit derived from the polyfunctional compound.

In embodiments, suitable reaction products can be non-toxic polymetal complexes that include calcium, magnesium, copper, zinc, aluminum, and/or silver constituents. Such calcium, magnesium, copper, zinc, aluminum, and/or silver reaction products include, but are not limited to water soluble compounds that contain calcium, magnesium, copper, zinc, aluminum, and/or silver. Non-limiting examples of water-soluble polymetal complexes include copper-zinc citrate, copper-silver citrate, silver-zinc citrate, copper-zinc oxalate, copper-silver oxalate, silver-zinc oxalate, copper-zinc tartarate, copper-silver tartarate, silver-zinc tartarate, copper-zinc malate, copper-silver malate, silver-zinc malate, copper-zinc succinate, copper-silver succinate, silver-zinc succinate, copper-zinc malonate, copper-silver malonate, silver-zinc malonate, copper-zinc maleate, copper-silver maleate, silver-zinc maleate, copper-zinc aspartate, copper-silver aspartate, silver-zinc aspartate, copper-zinc glutamate, copper-silver glutamate, silver-zinc glutamate, copper-zinc glutarate, copper-silver glutarate, silver-zinc glutarate, copper-zinc fumarate, copper-silver fumarate, silver-zinc fumarate, copper-zinc glucarate, copper-silver glucarate, silver-zinc glucarate, copper-zinc polyacrylic acid, copper-silver polyacrylic acid, silver-zinc polyacrylic acid, calcium-zinc aspartate, magnesium-calcium aspartate, magnesium-zinc aspartate, calcium-magnesium adipate, calcium-magnesium glutamate, calcium-magnesium malate, calcium-magnesium butanedioate, and combinations thereof.

In embodiments, copper, zinc, aluminum, and silver salts of organic multi carboxylic acids are suitable for use in accordance with the present disclosure. In embodiments, suitable salts can be doped such that the unit cell of the salt has zinc or silver constituents dispersed therein. Such zinc or silver constituents may either substitute another metallic constituent or fill a preexisting void in the unit cell.

In embodiments, suitable reaction products can be copper salts having zinc or silver constituents therein. For example, zinc or silver may either substitute a copper constituent or fill a preexisting void in the copper salt's unit cell. Suitable non-limiting examples of copper salts which may be used to form polymetallic complexes include copper (II) malonate and any hydrated form thereof such as copper (II) malonate dihydrate, copper (II) malonate trihydrate, and copper malonate tetrahydrate. Other suitable non-limiting examples of suitable copper salt active ingredients include copper citrate, copper oxalate, copper tartarate, copper malate, copper succinate, copper malonate, copper maleate, copper aspartate, copper glutamate, copper glutarate, copper fumarate, copper glucarate, copper polyacrylic acid, and combinations thereof. In embodiments, suitable copper salts can be doped such that the unit cell of the salt has zinc or silver constituents dispersed therein. Such zinc or silver constituents may either substitute a copper constituent or fill a preexisting void in the unit cell.

### Cu/Zn Malonate Embodiments

In embodiments, malonic acid may be reacted with salts containing copper and zinc constituents in an aqueous solution. It has been found that where the malonic acid, copper, and zinc constituents are present in at least about a 3:1:1 molar ratio, copper-zinc malonates may be produced in good yield and high crystalline purity.

Malonic acid refers to 1,3-propanedioic acid, a dicarboxylic acid with structure CH₂(COOH)₂ or:

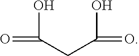

The ion form of malonic acid, as well as its esters and salts, are known as malonates. For example, diethyl malonate is ethyl ester of malonic acid. As used herein, the term copper-zinc malonate applies to any salt substances formed from malonic acid having copper and zinc constituents.

Suitable ingredients for the formation of copper-zinc malonates include malonic acid, one or more bases of copper and zinc, and water. In an aqueous reaction solution, suitable salt forms provide copper and zinc cations capable of bonding to malonate anions. Other suitable ingredients for the formation of copper-zinc malonates will include the replacement of bases of copper and zinc with the metallic form of copper and zinc. The elemental form of copper and zinc are known as copper and zinc metals and will be dissolved in the acidic water media as they react with malonic acid.

One or more salts containing copper and zinc constituents are present in amounts that will contact malonic acid in an aqueous solution. Suitable salts for making copper-zinc malonate compositions in accordance with this disclosure include metal salts containing complex-forming metal ions of copper and/or zinc. Non-limiting examples of suitable metal salts are copper (I) and (II) salts such as copper chloride, copper bromide, copper fluoride, copper nitrate, copper fluoroborate, copper sulfate, copper acetate, copper trifluoro acetate, copper stearate, copper octoate, copper methacrylate, copper malonate, copper benzoate; zinc salts such as zinc bromide, zinc chromate, zinc chloride, zinc stearate, zinc octoate, and zinc ethylhexoate. In embodiments, the aqueous solution may include one or more metallic salts, such as cupric carbonate (CuCO₃Cu(OH)₂), zinc carbonate (3Zn(OH)₂2ZnCO₃), metallic copper, metallic zinc, and combinations thereof. Basic salts such as basic zinc salts, basic copper salts, and combinations thereof are also suitable for use in accordance with the present disclosure. In embodiments, suitable metal basic salts are: copper (I) and (II) salts such as copper carbonate, copper oxide, and copper hydroxide; and zinc salts such as zinc carbonate, zinc oxide, and zinc hydroxide.

It should be understood that the listed salts are only a small portion of the salts suitable for use in accordance with the present disclosure. For example, inorganic salts are suitable provided that they provide copper and zinc cations when placed in an aqueous solution. Thus, the foregoing list of salts should be considered a non-limiting, illustrative list.

For carrying out the process, the reaction solution can be prepared by mixing the various ingredients in water where malonic acid and the salts may ionize and become more reactive. Water in the mixture is added in limited amounts sufficient to allow copper-zinc malonates to precipitate from solution upon formation. Accordingly, the reaction mixture is not so dilute as to prevent product precipitate formation. Where copper and zinc salts in the reaction mixture are insoluble and form dispersions (such as at cooler temperatures), mixing and heating steps can be applied to bring the reactants to about 40° C.-100° C. in order to force the reaction. As a result, reactant solubility may be enhanced through energy input such as microwave heating or addition of boiling water dissolver. The input of the energy may take place through any instrument capable of heating the aqueous reaction mixture. The copper-zinc malonate complexes formed in solution may be immediately separated so that their production can take place in a continuous process. Due to the short reaction time and the rapid crystallization of the copper-zinc malonate product, the conversion may be carried out continuously, and the recovery of the resultant solid product may take place by any conventional manner such as filtering, centrifugation, or sedimentation.

In the production of the reaction mixture, the concentration of the polyfunctional compound and that of the copper and zinc constituents may be pre-selected so that the total concentration of product formed exceeds the solubility equilibrium. This will result in product precipitating from solution in solid form for easy collection.

In embodiments, the final composition may be a deep blue crystal having good yield and substantial crystalline purity. Suitable copper-zinc malonate forms in accordance with the present disclosure include any salt formed from the neutralization of malonic acid by one or more copper containing molecules and one or more zinc containing molecules. Illustrative examples include salt formed by the neutralization of malonic acid by cupric carbonate (CuCO₃Cu(OH)₂), and zinc carbonate (3Zn(OH)₂ 2ZnCO₃) in an aqueous solution. Here copper may be added first, followed by zinc in order to obtain the salts of the present disclosure.

In embodiments, the copper-zinc malonates may be one or more ionic compounds formed by joining one or more independent copper molecules or ions and one or more independent zinc molecules or ions to a central unit by ionic bonds. For example, the copper-zinc malonate may be in the form of a trinuclear cation, where structurally independent copper and zinc hydrates are bridged by a central unit such as an octahedral diaquadimalonatocopper (II) unit. However, various coordination modes are possible depending on the source of the copper and zinc and synthesis conditions. In embodiments, the central unit malonate ion may be a multi-membered ring such as eight-membered ring, six-membered ring, and four-membered metalocycle for bridging or chelating functions between the copper and zinc constituents. Accordingly, the crystal structures of copper-zinc malonates can be very diverse, from ionic to three-dimensional polymers. In embodiments, the copper-zinc malonates can be found in several hydrate, and polymorphic forms. In embodiments, the process parameters are especially advantageous if the polyfunctional compound is added to excess in comparison to the metal counter cation constituents. Depending on the desired complex, the latter are added so that the molar ratio of polyfunctional compound to metal ions is approximately 3:2.

### Ca/Zn Malonate Embodiments

In embodiments, malonic acid may be reacted with salts containing calcium and zinc constituents in an aqueous solution. It has been found that where the malonic acid, calcium, and zinc constituents are present in at least about a 3:1:1 molar ratio, calcium-zinc malonates may be produced in good yield and high water solubility.

Suitable ingredients for the formation of calcium-zinc malonates include malonic acid, one or more bases of calcium, zinc, and water. In an aqueous reaction solution, suitable salt forms provide calcium and zinc cations capable of bonding to malonate anions.

One or more salts containing calcium and zinc constituents are present in amounts that will contact malonic acid in an aqueous solution. Suitable salts for making calcium-zinc malonate compositions in accordance with this disclosure include metal salts containing complex-forming metal ions of calcium and/or zinc. Non-limiting examples of suitable metal salts are salts such as calcium carbonate, calcium hydroxide, calcium sesquicarbonate, and calcium hydroxyl carbonate. In embodiments, the aqueous solution may include one or more metallic salts, such as calcium carbonate (CaCO₃), zinc carbonate (3Zn(OH)₂ 2ZnCO₃), and other combinations of the listed elements in basic form. Basic salts such as basic zinc salts, basic calcium salts, and combinations of other basic cationic salts are also suitable for use in accordance with the present disclosure. As described above, molar adjustments for suitable reactivity may be made to increase the water solubility of the complex by increasing the most water soluble cation and decreasing the most water insoluble component, accordingly.

It should be understood that the listed salts are only a small portion of the salts suitable for use in accordance with the present disclosure. For example, inorganic salts are suitable provided that they provide cations when placed in an aqueous solution. Thus, the foregoing list of salts should be considered a non-limiting, illustrative list.

For carrying out the process, the reaction solution can be prepared by mixing the various ingredients in water where malonic acid and the salts may ionize and become more reactive. Water in the mixture is added in limited amounts sufficient to allow calcium-zinc malonates to form in solution. Accordingly, the reaction mixture is not so dilute as to prevent product formation. Due to the short reaction time and the rapid solubilization of the calcium-zinc malonate product, the conversion may be carried out continuously, and the recovery of the resultant product may take place by any conventional manner such as filtering, centrifugation, or sedimentation.

In the production of the reaction mixture, the concentration of the polyfunctional compound and that of the calcium and zinc constituents may be pre-selected so that the total concentration of product formed doesn't exceeds the solubility equilibrium. This will result in concentrated solution for easy decanting from precipitated unreacted metallic carbonates.

### Embodiments of Compositions Containing the Polymetal Complex

In embodiments, the polymetal complex formed from the resulting reaction products may serve as active ingredients in compositions suitable for administration to the oral cavity. Such active ingredients may be combined with numerous ingredients to form a variety of products which may be capable of oral administration. The active ingredients in suitable toxicological compositions can be applied to the oral region or tissues of humans or other mammals. Such products may include a dermatologically or pharmaceutically acceptable carrier, vehicle or medium, for example, a carrier, vehicle, or medium that is compatible with the tissues to which they will be applied. Some non-limiting examples include water, saline, dextrose, oil-in-water, or water-in-oil emulsions. Some additional examples are described in REMINGTON'S PHARMACEUTICAL SCIENCES (Mack Publishing Company). The term “dermatologically or pharmaceutically acceptable,” as used herein, means that the compositions or components thereof so described are suitable for use in contact with these tissues or for use in subjects in general without undue toxicity, incompatibility, instability, allergic response, and the like. In embodiments, compositions in accordance with the present disclosure can contain any ingredient conventionally used in foods, cosmetics, and/or pharmacology. In embodiments, active ingredients may be formulated to provide crystals in solution, as well as solid forms.

In embodiments, products containing a reaction product in accordance with the present disclosure as an active ingredient can be in the form of solutions, emulsions (including microemulsions), suspensions, creams, lotions, gels, powders, foams, mouthwashes, aerosols, sprays, or other typical solid or liquid compositions used for treatment of oral disorders. Such compositions may contain, in addition to the reaction product in accordance with this disclosure, other ingredients typically used in such products, such as pharmaceutically active agents, moisturizers, hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents, buffers, protectants, pH regulators, chelating agents, propellants, counter-irritants, humectants, lubricants, astringents, conditioners, darkening or lightening agents, glitter, mica, minerals, silicones, polyphenols, sunblocks, phytomedicinals, and combinations thereof.

The term “pharmaceutically active agents” is meant to have the broadest interpretation as to any therapeutically active substance which is delivered to a living organism to produce a desired and often beneficial result. Some not limiting examples include antibiotics, antiseptics, anesthetics, muscle relaxants, antihistamines, decongestants, antimicrobial agents, anti-viral agents, anti-fungal agents, antimalarials, amebicides, antituberculosal agents, antiretroviral agents, leprostatics, antiprotazoals, antihelmitics, antibacterial agents, steroids, hematopoietic agents, antiplatelet agents, anticoagulants, coagulants, thrombolytic agents, hemorrheologic agents, hemostatics, plasma expanders, hormones, sex hormones, uterine-active agents, bisphosphonates, antidiabetic agents, glucose-elevating agents, growth hormones, thyroid hormones, inotropic agents, antiarrhythmic agents, calcium channel blockers, vasodilators, sympatholytics, antihyperlipidemic agents, vasopressors, angiotensin antagonists, sclerosing agents, anti-impotence agents, urinary alkanizers, urinary acidifiers, anticholinergics, diuretics, bronchodilators, surfactants, antidepressants, antipsychotics, antianxiety agents, sedatives, hypnotics, barbiturates, antiemetic agents, analgesics, stimulants, anticonvulsants, antiparkinson agents, proton pump inhibitors, H₂-antagonists, antispasmodics, laxatives, antidiarrheals, antiflatulents, digestive enzymes, gallstone solubilizing agents, antihypertensive agents, cholesterol-lowering agents, radiopaque agents, immune globulins, monoclonal antibodies, antibodies, antitoxins, antivenins, immunologic agents, anti-inflammatory agents, antineoplastic agents, alkylating agents, antimetabolites, antimitotic agents, radiopharmaceuticals, vitamins, herbs, trace elements, amino acids, enzymes, chelating agents, immunomodulatory agents and immunosuppressive agents, wound healing agents, adhesives, sealants, blood products, blood components, ultraviolet absorbers, ultraviolet stabilizers, photochromic agents, proteins, polysaccharides, peptides, genetic material, immunological agents, anti-colonization agents, diagnostic agents, imaging agents, antacids, and combinations thereof.

As an illustrative example, products can be formulated to contain copper-zinc malonate in amounts from about 0.001 to about 25% by weight of the total composition. In embodiments, products can be formulated to contain copper-zinc malonate in an amount from about 0.05 to about 10% by weight of the total composition. In other embodiments, the amount of copper-zinc malonate is from about 0.1 to about 5% by weight of the total composition. Here, the copper-zinc malonate present may be in a pharmaceutically acceptable salt form. Other active ingredients may be provided in the formulations at the same concentrations.

The particular active ingredient or ingredients employed, and the concentration in the compositions, generally depends on the purpose for which the composition is to be applied. For example, the dosage and frequency of application can vary depending upon the type and severity of the oral disorder.

Similarly, the pH of the compositions may vary according to the form of the composition, the ingredients contained therein and the type of tissue the composition is contacting. In embodiments, the pH of the compositions may range from about 4.0 to about 10.0. In embodiments, the pH of the composition may range from about 4.5 to about 8.0, and in some embodiments, the pH of the composition may range from about 5.5 to about 6.0.

In embodiments, the compositions may include the polymetal complex and at least one pharmaceutically active agent known to treat disorders of the oral region. In one example, the composition may include a polymetal complex and hydrocortisone, a drug commonly found in ointments and creams. In other embodiments, the composition may include a polymetal complex and an anesthetic such as dibucaine, benzocaine, lidocaine, and the like. In still other embodiments, the compositions may include a polymetal complex and a pain reliever such as acetaminophen, ibuprofen, codeine, and the like. Compositions which include various combinations of pharmaceutically active agents are also envisioned.

It has also been discovered that the compositions which contain a polymetal complex of the present disclosure are useful in causing varying levels of vasoconstriction. Such an effect may be useful in many oral disorders. Moreover, the vasoconstrictive effect of the present compositions decrease the rate at which the body is able to clear the composition by local blood supply, thereby allowing the composition to remain at the site of application longer which increases the rate and depth of tissue penetration of the composition. In embodiments, the compositions of the present application may be combined with other vasoconstrictive agents to further enhance the effect of the polymetal complex. In still other embodiments, the compositions of the present application may be combined with vasodilating agents thereby decreasing the effect of the polymetal complex.

In embodiments, the compositions described herein may be incorporated into mouthwash formulations for oral administration. The polymetal complexes may be combined with any known mouthwash base material.

The mouthwashes and oral rinses may be formed by any dissolving ingredients in stable solutions. In embodiments, the composition including the polymetal complex may be mixed with mouthwashes and oral rinse solutions. The composition may be a gel or liquid.

In other embodiments, the compositions described herein may be incorporated into oral products. Generally, oral products are liquid compositions, solutions, emulsions or suspensions, gels, or ointments which may contain additional ingredients such as thickeners, preservatives, pH regulators, thickeners, and active agents. The oral products described herein may include from about 0.001mg to about 10 mg of polymetal complex per dose.

In still other embodiments, the compositions described herein may be incorporated into oral foams. Oral foams may have a polymetal complex content from about 0.001 mg/dose to about 10 mg/dose. In addition, foams may also include: traditional solubilizers, such as purified water and glycerin; emulsifiers, such as polysorbate 20 and alcohol with chelating agents, such as ethylenediaminetetraacetic acid, also in the form of sodium salt; and preservatives, acidifying buffers, such as phosphoric acid and monobasic sodium or potassium phosphate; propellants, such as hydrocarbons, e.g., isobutane, or fluorocarbons, e.g., dichlorodifluoromethane and dichlorotetrafluoroethane, or hydrochlorofluorocarbons or hydrofluorocarbons. As concerns the pharmaceutical formulation, oral foams and sprays—compared with mouthwashes—have a lower water content and contain propellants, which are indispensable for dispensing the dose of drug to be administered.

Treatments in accordance with the present disclosure contact the oral region, such as the oral mucosa tissue, with one or more active ingredients such as those containing copper, zinc, and/or silver in an effective amount to improve the undesirable disorder. In embodiments, compositions containing a polymetal complex in accordance with the present disclosure are applied externally to the specialized mucosa or to the lining mucosa of the oral mucosa. In embodiments, subjects are treated by administering one or more copper-zinc malonates to a subject's oral region, such as the lips (e.g., the mucosal surfaces of the lips). In embodiments, subjects suffering from an oral disorder are treated by inserting or applying to tissue, one or more salts in accordance with the present disclosure. The active ingredient is applied until the treatment goals are obtained. However, the duration of the treatment can vary depending on the severity of the condition. For example, treatments can last several days or weeks depending on whether the goal of treatment is to reduce or eliminate the condition.

In embodiments, a copper-zinc carboxylic acid salt having copper and zinc cations in the same molecule is applied to oral tissue.

In treatment embodiments, the compositions and methods in accordance with the present disclosure can be combined with other oral and systemic treatment systems. For example, the polymetallic salt complexes can be applied to the oral region of a subject in combination with another antifungal or antibiotic for systemic ingestion as a treatment option. The active ingredients and formulations in accordance with the present disclosure may either be incorporated into other product formulations, or applied to the oral region before, after, and/or during other treatments.

### EXAMPLES

The following non-limiting examples of compositions suitable for use in the treatment of oral disorders in accordance with the present disclosure were formulated.

### Example 1

A vasoconstrictive gel was formulated having the constituents:

| | |
|---|---|
| Water (Purified) | 69.28 |
| Propylene glycol | 2.00 |
| Copper(2) Zinc Malonate | 0.12 |
| Phenylephrine Hydrochloride | 0.25 |
| Water | 1.00 |
| Potassium Sorbate | 0.30 |
| Phenoxyethanol | 1.00 |
| Glycerin | 20.00 |
| Hydroxyethylcellulose (250H) | 1.00 |
| Flush - Water (Purified) | 5.00 |
| Sodium Hydroxide (10% w/v) | 0.05 |

### Example 2

An antifungal emulsion was formulated having the following constituents:

| | |
|---|---|
| Petrolatum | 58.28 |
| Glycerine | 22.00 |
| Sorbitol (70% sol'n) | 5.00 |
| Dimethicone | 2.00 |
| Nystatin | 1.00 |
| Polysorbate 20 | 0.1 |
| Polyacrylate-13 | 0.9 |
| Copper/Zinc Hyaluronate | 0.25 |
| Potassium Sorbate | 0.30 |
| Phenoxyethanol | 1.00 |
| Water (Purified) | 7.17 |
| Sodium Hydroxide (10% w/v) | Q.S. |

### Example 3

An oral gel was formulated as follow:

| | |
|---|---|
| Water (Purified) | 61.38 |
| Carbamide peroxide | 10.000 |
| Copper(2)/Zinc/Sc Glutamate | 0.06 |
| Water | 1.000 |
| Potassium Sorbate | 0.3000 |
| Phenoxyethanol | 1.0000 |
| Glycerin | 20.0000 |
| Hydroxyethylcellulose (250H) | 1.0000 |
| Flush - Water (Purified) | 5.2500 |
| Sodium Hydroxide (10% w/v) | 0.01 |

### Example 4

An oral rinse was formulated as follow:

| | |
|---|---|
| Water (Purified) | 69.28 |
| Propylene glycol | 2.0000 |
| Calcium/Zn Malicate | 0.242 |
| Menthol | 0.100 |
| Water | 2.0000 |
| Potassium Sorbate | 0.3000 |
| Phenoxyethanol | 1.000 |
| Glycerin | 20.0000 |
| Flush - Water (Purified) | 5.0000 |
| Sodium Hydroxide (10% w/v) | 0.078 |

### Example 5

A lipstick was prepared including: Glycerin 81.45%, Sodium Stearate 8.5%, Ca-Zn Malonate 0.05%, and Benzyl Alcohol 10%.

### Example 6

An antibiotic lip ointment was prepared with: Petrolatum USP 99.0% and Cu-Zn Bacitracin 1.0%.

While several embodiments of the disclosure have been described, it is not intended that the disclosure be limited thereto, as it is intended that the disclosure be as broad in scope as the art will allow and that the specification be read likewise. Therefore, the above description should not be construed as limiting, but merely as exemplifications of embodiments. Those skilled in the art will envision other modifications within the scope and spirit of the claims appended hereto.

1-20. (canceled)

21. A method of treating signs of chronological aging or environmental damage to lips or skin of a perioral region of a subject, the method comprising administering a composition including an effective amount of a polymetal complex to an oral cavity of the subject.

22. The method of claim 21, wherein administering the composition includes contacting tissue within the oral cavity with the polymetal complex that is a reaction product of a polyfunctional compound with two or more elements selected from calcium, magnesium, copper, silver, gold, aluminum, scandium, titanium, vanadium, chromium, manganese, iron, cobalt, nickel, zinc, gallium, yttrium, zirconium, nobium, molybdenum, technetium, ruthenium, rhodium, palladium, germanium, cadmium, indium selenium, and tin.

23. The method of claim 22, wherein the polyfunctional compound is a polyfunctional acid.

24. The method of claim 22, wherein the polyfunctional compound is selected from the group consisting of maleic acid, fumaric acid, citraconic acid, itaconic acid, glutaconic acid, phthalic acid, isophthalic acid, terephthalic acid, cyclohexane dicarboxylic acid, citric acid, succinic acid, adipic acid, sebacic acid, azealic acid, malonic acid, dodecanedioic acid, 1,18-octadecanedioic acid, dimer acids, alkenyl succinic acids, and hyaluronic acid.

25. The method of claim 22, wherein the polyfunctional compound is a polyfunctional amine.

26. The method of claim 22, wherein the polyfunctional compound is an amino acid.

27. The method of claim 21, wherein the composition is an oral product selected from the group consisting of solutions, emulsions, suspensions, gels, and ointments, and wherein administering the composition includes applying the oral product inside the oral cavity.

28. The method of claim 21, wherein the composition is a mouthwash, and wherein administering the composition includes rinsing the oral cavity with the mouthwash.

29. The method of claim 21, wherein the composition is an oral foam, and wherein administering the composition includes dispensing the oral foam inside the oral cavity.

30. The method of claim 21, wherein administering the composition includes delivering from about 0.001 mg to about 10 mg of the polymetal complex per dose.

31. The method of claim 21, wherein administering the composition includes contacting oral mucosa tissue with the composition to treat wrinkles around a mouth of the patient.

32. A method comprising
administering a composition including an effective amount of a polymetal complex to an oral cavity of a subject exhibiting signs of chronological aging or environmental damage to lips or skin of a perioral region,
wherein the polymetal complex is a reaction product of a polyfunctional compound with two or more elements selected from calcium, magnesium, copper, silver, gold, aluminum, scandium, titanium, vanadium, chromium, manganese, iron, cobalt, nickel, zinc, gallium, yttrium, zirconium, nobium, molybdenum, technetium, ruthenium, rhodium, palladium, germanium, cadmium, indium selenium,
the polymetal complex administered in an amount effective to reduce signs of chronological aging or environmental damage to lips or skin of a perioral region of a subject.

33. The method of claim 32, wherein administering the composition includes contacting tissue within the oral cavity with the polymetal complex that is a reaction product of
(i) a polyfunctional acid selected from the group consisting of maleic acid, fumaric acid, citraconic acid, itaconic acid, glutaconic acid, phthalic acid, isophthalic acid, terephthalic acid, cyclohexane dicarboxylic acid, citric acid, succinic acid, adipic acid, sebacic acid, azealic acid, malonic acid, dodecanedioic acid, 1,18-octadecanedioic acid, dimer acids, alkenyl succinic acids, and hyaluronic acid and
(ii) either (a) calcium and magnesium or (b) copper and zinc.
